(19) United States
(12) Patent Application Publication
(10) Pub. No.: US 2022/0076794 A1
(43) Pub. Date: Mar. 10, 2022

(54) SYSTEMS AND METHODS FOR REQUESTING AND RETRIEVING MEDICAL RECORDS BETWEEN DISPARATE MEDICAL PROVIDERS

(71) Applicant: MEDICOM TECHNOLOGIES INC., Raleigh, NC (US)

(72) Inventors: CHRIS WOODLIEF, RALEIGH (US); BRENT GOLDSTEIN, RALEIGH (US); CHASE BALLARD, RALEIGH (US)

(21) Appl. No.: 17/466,435

(22) Filed: Sep. 3, 2021

Related U.S. Application Data
(60) Provisional application No. 62/706,709, filed on Sep. 4, 2020.

(51) Int. Cl. G16H 10/60; G06Q 10/10; H04L 29/06

(52) U.S. Cl. CPC G16H 10/60; H04L 63/061; G06Q 10/1095

(57) ABSTRACT

The present invention relates, in general, to systems and methods that allow medical providers to request and send medical records, and in particular medical imaging studies, across disparate medical networks, and between medical providers that are may or may not be part of a same or affiliated medical network, thereby eliminating the need for physical media to be created and physically delivered.

### CROSS-REFERENCE TO RELATED APPLICATION

This application claims the benefit of U.S. Provisional Patent Application No. 62/706,709 entitled “SYSTEM AND METHOD FOR REQUESTING AND RETRIEVING MEDICAL RECORDS BETWEEN DISPARATE MEDICAL PROVIDERS” filed on Sep. 4, 2020, which is commonly owned, the disclosure of which is incorporated herein by reference in its entirety.

### BACKGROUND

### Field of the Invention

The present invention relates, in general, to systems and methods that allow medical providers to electronically search for, and submit requests for, prior medical records from other medical providers that are may not be part of a same or affiliated medical network, thereby eliminating the need for physical media to be created and physically delivered, as well automating the reconciliation of prior medical records that may be received from external medical providers.

### Description of Related Art

For medical providers, such as radiologists, the ability to receive all pertinent prior medical imaging, diagnostic reports, and records for a patient is critical so that prior data can be compared or referenced against the patient's current imaging studies. Medical imaging is absolutely necessary when tracking the progress of an ongoing illness. For example, mammogram, MRIs, and CT scans allow the physician to monitor the effectiveness of treatment and adjust protocols as necessary. The detailed information generated by medical imaging provides patients with better, more comprehensive care.

Medical providers routinely need to locate and retrieve as much of a patient's prior medical records, especially when the patient is a new patient, or when the patient is being treated by multiple medical providers across different medical facilities and practices. Providers typically needs to reconcile the external patient demographics associated with the prior medical records, such as the patient name, patient identification, and the like, with the requesting medical provider's internal information systems.

Typically, providers have the challenging task of locating prior medical records based on limited or incorrect information. Such information is obtained during the patient intake process or during scheduling the patient for a new visit, such as for a diagnostic imaging exam. This requires the provider to oftentimes make several phone calls to different external medical providers to determine (1) if any prior medical records exist, and (2) if so, which prior medical records are relevant to the upcoming visit. In addition, due to Health Insurance Portability and Accountability Act of 1996 (HIPAA) as well as policies that may be in place by external medical providers, a phone call may not be a preferred method for such records requests, and the provider typically must manually fill out a records request and fax the request to the external medical provider.

Compounding the complexity of this problem, patients may not always remember with whom, when or where their previous medical visits were, or if any prior medical records exist with any external medical providers.

If and when the prior medical records are ultimately located, they are sent to the requesting medical provider, where they must then be reconciled in order to be made available for review by the medical provider. If the prior medical records are not reconciled property, a medical provider may be left without all of the information relevant to a patient, or may have to spend time manually locating data.

The ability to review prior medical records in a timely manner is critical. Delays in identifying and retrieving prior medical records can impede the review new imaging studies and providing interpretations, which can ultimately impact patient care.

The above challenges are common to medical providers of all sizes and specialties, as providers traditionally rely on the use of phone calls and paper-based fax for submitting records requests for prior medical records. In addition, providers must currently accept that timely cooperation with external medical providers may not always be that case, and that their ability to deliver optimal patient care could be compromised.

Thus, there is a need for an electronic system and method that allows a requesting medical provider to search for, request, receive, and reconcile prior medical records in an efficient and timely manner, without the use of traditional methods such as phone calls and paper-based fax requests.

### SUMMARY

In an embodiment, the invention relates to a method for retrieving a medical record from a remote database, comprising: scheduling a patient for an appointment by a first medical provider; determining, by the first medical provider, if the patient has a prior medical record from a second medical provider; transmitting, by the first medical provider, an electronic record request to the second medical provider via a data transfer network, wherein the electronic record request includes a request for the prior medical record; transmitting, by the second medical provider, the prior medical record to the first medical provider; receiving, by the first medical provider, the prior medical record; and updating, by the first medical provider, a prior patient identification number associated with the prior medical record.

In another embodiment, the invention relates to a system for retrieving prior medical records for a patient from a remote database, comprising: a server communicatively coupled to both a first medical provider and a second medical provider; a first local server coupled to the first medical provider, wherein the first local server is an instance of the server; a second local server coupled to the second medical provider, wherein the second local server is an instant of the server; a first medical records database coupled to the first local server; a second medical records database coupled to the second local server; and a portal capable of being executed on the first local server and the second local server, wherein the first local server is capable of automatically retrieving patient demographic information from the first medical records database based on the patient identification number, and wherein the first local server generates an electronic record request using at least one of the patient demographic information and record request information, and wherein the first local server transmits the electronic record request to the second local server via the server.

In yet another embodiment, the invention relates to a system for retrieving a prior medical record for a patient from a remote database, comprising: a patient scheduling system communicatively coupled to a first medical provider, wherein the patient scheduling system is capable of generating an appointment date; a server communicatively coupled to both the first medical provider and a second medical provider; a first local server coupled to the first medical provider, wherein the first local server is an instance of the server; a second local server coupled to the second medical provider, wherein the second local server is an instant of the server; a first medical records database coupled to the first local server; a second medical records database coupled to the second local server; and a portal capable of being executing on the first local server and the second local server, wherein the first local server is capable of automatically retrieving patient demographic information from the first medical records database based on a patient identification number, and wherein the first local server generates an electronic record request using the patient demographic information, record request information, the appointment date, and a unique identification number, and wherein the first local server transmits the electronic record request to the second local server via the server.

### BRIEF DESCRIPTION OF THE DRAWINGS

These and other embodiments of the disclosure will be discussed with reference to the following exemplary and non-limiting illustrations, in which like elements are numbered similarly, and where:

FIG. 1 is a network architecture diagram of a system for transmitting and fulfilling electronic medical requests across disparate medical providers, according to an embodiment of the invention;

FIG. 2 is a flowchart illustrating the steps of reconciling prior medical records received from an external medical provider by a requesting medical provider, according to an embodiment of the invention;

FIG. 3 is an exemplary interface for creating an electronic record request, according to an embodiment of the invention;

FIG. 4 is an exemplary interface for displaying an alert if the patient ID does not match any patient records, according to an embodiment of the invention;

FIG. 5 is an exemplary interface for displaying multiple patient records which match a patient ID, according to an embodiment of the invention;

FIG. 6 is an exemplary interface for selecting recipients for an electronic record request, according to an embodiment of the invention;

FIG. 7 is an exemplary interface for confirming an electronic record request, according to an embodiment of the invention;

FIG. 8 is an exemplary interface for displaying a success status of an electronic record request, according to an embodiment of the invention;

FIG. 9 is an exemplary interface for displaying an error status of an electronic record request, according to an embodiment of the invention;

FIG. 10 is an exemplary interface displaying an inbox for received electronic record requests, according to an embodiment of the invention;

FIG. 11 is an exemplary interface displaying a selected electronic medical request, according to an embodiment of the invention;

FIG. 12 is an exemplary interface for uploading files to fulfill an electronic medical request, according to an embodiment of the invention;

FIG. 13 is an exemplary interface displaying an outbox for fulfilled electronic record requests, according to an embodiment of the invention;

FIG. 14 is an exemplary interface to confirm a transmission of selected prior medical records to a requesting medical provider, according to an embodiment of the invention; and

FIG. 15 is an exemplary interface for configuring privileges by an external medical provider, according to an embodiment of the invention

### DEFINITIONS

The following definitions are meant to aid in the description and understanding of the defined terms in the context of the present invention. The definitions are not meant to limit these terms to less than is described throughout this application. Such definitions are meant to encompass grammatical equivalents.

As used herein, the term “medical record” can refer to, for example, any patient data, patient charts, electronic medical records, medical imaging studies, medical and diagnostic images, diagnostic reads and reports, diagnostic notes, medical opinions, medical test and laboratory results, surgical history, family medical histories, medication lists, medical allergies, social histories and habits (such as, for example, drug, tobacco and alcohol use), immunization histories, clinical information, growth and development histories, medical encounter histories, physical examination observations, medical progress notes, dietary information, fitness and activity data, travel history, contact tracing records, and the like.

As used herein, the term “provider” can refer to, for example, a physician (including, but not limited to, a radiologist, surgeon, primary care physician, and a medical specialist), a physician assistant, a nursing professional, a medical laboratory technician, medical clinics, hospitals, health insurance providers, diagnostic sites, imaging sites, pharmacies, practice administrators, Medical Imaging Management staff, and the like. A “provider” can further be an individual patient, an academic institution, a government research laboratory, a non-profit entity, or a for-profit entity, such as a pharmaceutical, health insurance, biotechnology, wearable device, physiological monitoring, or medical device company.

As used herein, the term “data exchange interface” can refer to, for example, an interface, standard, specification and/or protocol that allows for disparate systems to share data, including, but not limited to, the Health Level Seven (HL7) standard, the Fast Healthcare Interoperability Resources (FHIR) standard, the HPRIM standard, the Continuity of Card Record (CCR) standard, the Continuity of Care Document (CCD) specification, the xDT data exchange format, the Arden syntax, an application programming interface (API), and direct access protocols for electronic health records systems, and the like.

### DETAILED DESCRIPTION

It should be understood that aspects of the invention are described herein with reference to the figures, which show illustrative embodiments. The illustrative embodiments herein are not necessarily intended to show all embodiments in accordance with the invention, but rather are used to describe a few illustrative embodiments. Thus, aspects of the invention are not intended to be construed narrowly in view of the illustrative embodiments. In addition, although the invention is described with respect to its application for the transfer of medical information containing medical imaging studies and associated radiology reports in the Digital Imaging and Communications in Medicine (DICOM) format, it is understood that the system could be implemented in any setting where the transfer of any type or form of medical, patient, and/or personally identifying data may be useful.

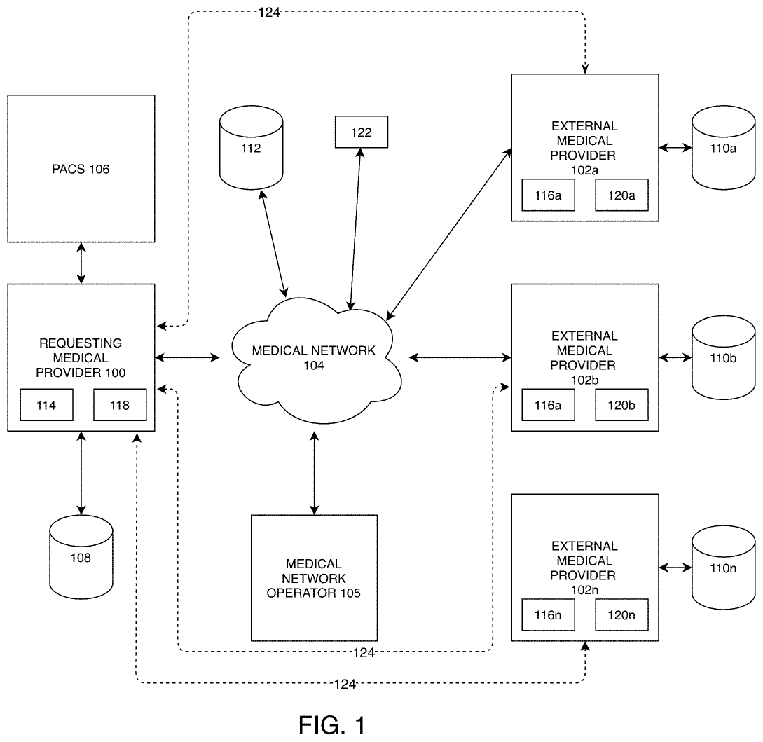

FIG. 1 is a network architecture diagram of a system for transmitting and fulfilling electronic medical requests across disparate medical providers, according to an embodiment of the invention. In an embodiment, a requesting medical provider 100 is communicatively coupled to at least external medical provider 102 via a medical network 104. The medical network 104 can be a restricted network operated by a medical network operator 105, such that the medical providers 100, 102 are registered with the medical network 104, and have a relationship with the medical network operator 105.

The term “external medical provider” as used herein refers to a medical provider which receives electronic medical requests. It is noted that an external medical provider can also be a requesting medical provider, and vice versa.

In an embodiment, the medical network 104 includes computing hardware, software, and/or services that facilitate the searching of medical records associated with the medical providers 100, 102. In an embodiment, the medical network 104 is configured as a server, local server, or a distributed server that can store data, as well as execute programs, algorithms, scripts, and applications.

In an embodiment, the medical network 104 is a proprietary network that facilitates direct peer-to-peer data transfer between the medical providers 100, 102 as described in more detail in commonly owned application Ser. No. 16/281,409, entitled, METHODS AND SYSTEMS FOR TRANSFERRING SECURE DATA AND FACILITATING NEW CLIENT ACQUISITIONS”, the contents of which are hereby incorporated by reference in its entirety.

In an embodiment, the medical providers 100, 102 are, or are coupled to, a medical facility, hospital, outpatient clinic, diagnostic imaging center, diagnostic imaging station, and the like. In an embodiment, the requesting medical provider 100 is communicatively coupled to a picture archiving and communication system (PACS) 106. The requesting medical provider 100 can be remote from, or locally coupled to, the PACS 106, and the requesting medical provider 100 can be communicatively coupled to multiple local or distributed PACS (not shown).

Furthermore, each of the external medical providers 102 are communicatively coupled to respective PACS (not shown).

In an embodiment, the medical providers 100, 102a-n are communicatively coupled to respective computing hardware and software, and can be coupled to respective databases 108, 110a-n that contain medical records, as shown in FIG. 1. In an embodiment, the medical network operator 105 can be coupled to a database 112.

In an embodiment, the database 112 can be configured to store replicated versions of data stored on the databases 108, 110. The database 112 can be updated or refreshed to reflect real-time changes to each database 108, 110, or the database 112 can be updated or refreshed on a periodic basis, such as daily, weekly, monthly, etc. In an embodiment, the database 112 can be updated or refreshed based on a triggering event, such as for example, a request for medical records being received from a requesting medical provider 100 by the medical network 104.

Each database 108, 110, and 112 can be locally stored and managed by its respective associated entity, and each database 108, 110, and 112 can be distributed or cloud-based, and located remotely from its respective associated entity, such as on a remote server provided by Amazon Web Services® or the like. In an embodiment, each database 108, 110, and 112 can be a relational database, a SQL database, an object-oriented database, a centralized database, or a distributed database, such as a cloud-based database or a blockchain-based database stored across a distributed ledger. In an embodiment, the databases 108, 110 and 112 are capable of storing data and files in the DICOM format.

In an embodiment, each medical provider 100, 102a-n is configured to run a respective local server 114, 116a-n that is operated, managed, provided, and/or developed by the medical network operator 105. In an embodiment, the local servers 114, 116 can be servers which are configured as a virtual instances of medical network server 122, and can be executed on virtual machines or virtual servers. The local servers 114, 116 can be physical, virtual, or cloud-based servers, and they can also each have a distributed server architecture. In an embodiment, the local servers 114, 116 can include DICOM standard functionality, and is configured to interface with a patient scheduling system 118, such as, for example, an EHR system. The local servers 114, 116 are configured to interface with each respective patient scheduling system 118, 120 by, for example, a data exchange interface, such as the HL7 standard, the FHIR standard, the HPRIM standard, the CCR standard, the CCD specification, the xDT data exchange format, the Arden syntax, an API, direct access to an EHR and/or associated databases, and the like.

In an embodiment, the requesting medical provider 100 can enter a patient appointment into a patient scheduling system 118 that is integrated with the EHR system via a portal (i.e., such as in a FHIR interface compatible EHR or in an EHR with API access). In an embodiment, the patient appointment can be generated remotely and electronically entered or pushed into the patient scheduling system 118, which can be integrated into, or coupled with, for example, an EHR, a physician referral system, a laboratory testing ordering system, and the like. In an embodiment, the patient scheduling system 188 is capable of generating an appointment date for a patient.

In this embodiment, the local servers 114, 116 can leverage the FHIR interface in order to (a) obtain accurate patient demographics from a system that is considered a source of truth; and (b) enable the attachment of data that may not reside in databases 108, 110, or any other DICOM storage archive (e.g., archives or databases for laboratory testing results, prescriptions, clinical research data, and the like) in response to an electronic record request.

Additionally, an automated federated search process as fully described in described in commonly owned U.S. patent application Ser. No. 17/001589, filed on Aug. 24, 2020, and entitled, “SYSTEMS AND METHODS FOR FEDERATED SEARCHING AND RETRIEVAL OF MEDICAL RECORDS ACROSS DISPARATE DATABASES”, which is hereby incorporated by reference in its entirety, can be utilized as a primary attempt to obtain prior medical records from an external medical provider 102. The electronic record request functionality described herein may serve as a fallback or secondary mechanism if an automated federated search query yields no results or encounters an error.

In an embodiment, each local server 114, 116 can function as an edge compute server which allows for distributed processing of data by physically being closer to each respective database 108, 110 and/or patient scheduling system 118, 120.

In another embodiment, the functions of each local server 114, 116 can be executed by the medical network 104. In this embodiment, the medical network 104 includes a medical network server 122. In an embodiment, the medical network server 122 can be distributed across multiple computing resources within the medical network 104, or can be a dedicated computing resource.

In an embodiment, medical providers 100, 102 can be communicatively coupled to the medical network 104 via communication links (denoted by arrows in FIG. 1). These communication links may be any type of communication links suitable to allow interaction between the medical providers 100, 102, as well as with the medical network 104 and medical network operator 105. For example, the communication links may each be a wired network, a wireless network, or any combination thereof. Further, communication links may include a distributed computing network, an intranet, a local-area network (LAN) and/or a wide-area network (WAN or WLAN), or any combination thereof. For example, the LAN may make use of WIFI in its many variations and the WAN may make use of broadband, cellular and/or satellite networks using technologies including, but not limited to, CDPD, CDMA, GSM, PDC, PHS, TDMA, FLEX, ReFLEX, iDEN, TETRA, DECT, DataTAC, Mobitex, EDGE and other 2G, 3G, 4G, 5G and LTE technologies. However, those of ordinary skill in the art will appreciate that the communication links are not limited thereto. In another embodiment, the communication links may each include ethernet, Firewire, parallel, serial, or USB connections, or short-range communication protocols such as Bluetooth, infrared, Zigbee, and the like.

In an embodiment, while the medical network 104 facilitates communication of an electronic record request between the medical providers 100, 102, the actual transmission of medical records can occur via encrypted, secure peer-to-peer communication channels 124 which have been fully described in commonly owned U.S. patent application Ser. No. 15/361,319, filed on Nov. 25, 2016, and entitled, “METHODS AND SYSTEMS FOR PROVIDING SECURE AND AUDITABLE TRANSFER OF ENCRYPTED DATA BETWEEN REMOTE LOCATIONS”, which is hereby incorporated by reference in its entirety.

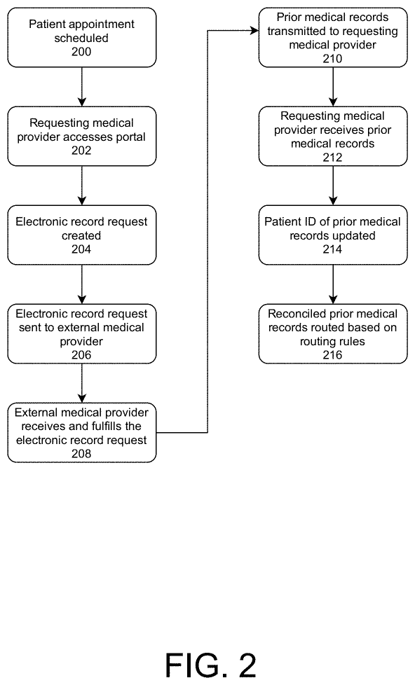

FIG. 2 is a flowchart illustrating the steps of reconciling prior medical records received from an external medical provider 102 by a requesting medical provider 100, according to an embodiment of the invention. In step 200, a patient schedules an appointment with the requesting medical provider 100. In an embodiment, the patient can utilize patient portal software managed, operated, or provided by the requesting medical provider 100 or the medical network operator 105. The patient portal software can be an online website, or mobile application, that is connected to the patient's electronic health records, and which is centrally focused on providing patient access to health data. The patient portal software can allow patients to access lab results, physician notes, their health histories, discharge summaries, immunizations, travel histories, and the like, as well as schedule medical consultations, visits, and procedures. The patient portal software can be, for example, MyChart® developed by Epic Systems Corporation, the Patient Portal and Healow® App developed by eClinicalWorks LLC, the athenaCommunicator® developed by AthenaHealth, Inc., and similar patient portal software developed by Accenture Federal Services plc, Allscripts Healthcare Solutions, Inc., Cerner Corporation, Computer Programs and Systems Inc., InteliChart LLC, and the like.

In another embodiment, the patient can utilize a patient scheduling system to create an appointment. The patient scheduling system can include, but is not limited to an electronic health record (EHR) system, electronic medical record system (EMR), a hospital information system (HIS), a Computerized Physician Order Entry systems (CPOE), and Integrating the Healthcare Enterprise Order Placer (IHE OP), a PACS, a Radiology Information System (RIS), a medical practice management system, a private patient scheduling system, a physician referral system, and the like.

In an embodiment, during or after scheduling the appointment, the patient can input additional details or notes for the requesting medical provider 100, such as details about external medical providers 102 which the patient is being treated by, or has been treated by in the past.

In an embodiment, the requesting medical provider 100 asks the patient if the patient has any prior visits to any external providers, such as prior medical imaging performed at other medical facilities.

In step 202, the requesting medical provider 100 can access a web-based or mobile-based portal to search for prior medical records located with external medical providers 102, and can generate electronic record requests for such prior medical records. In an embodiment, the portal can be executed by the local server 114, and can be managed, developed, or otherwise provided by the medical network 104 and or medical network operator 105. The portal can be a secure website accessed via a Uniform Resource Location (URL) using a browser on a computing device. In an embodiment, the computing device can be a PACS station, a medical workstation, and the like. In another embodiment, the portal can be a downloadable software application that is executed on a computing device, such as a mobile device, laptop computer, desktop computer, tablet computer, smartphone, and the like.

In an embodiment, prior to being able to access the portal, the provider must enter credentials, such as a login and password, or other indicia that verifies their identity. The credentials can include a user's mobile device number, login, password, e-mail address, phone number, account number, personal identification number (PIN), name, driver's license number, social security number, date of birth, employee number, and/or a unique account identification code previously provided to the administrator by an authorizing entity, such as an employer or the medical network operator 105. In another embodiment, the credentials can be biometric, such as a fingerprint, iris, facial, or voice scan. In yet another embodiment, the credential can be a gesture input by the user, such as a on a touchscreen or touchpad.

The portal can be stored on, or executed from, for example, the medical network 104, the medical network server, and/or the local servers 114, 116, and the portal allows an administrator or staff at the medical providers 100, 102 to configure various settings and criteria for searching and generating electronic record requests, as well as permissions and privileges for other medical providers in terms of searching, viewing, and requesting medical records. The portal also allows the medical providers 100, 102 to configure routing rules for the receipt and reconciliation of prior medical records.

In step 204, the requesting medical provider 100 can create an electronic record request for retrieving prior medical records for the patient, where the request includes a unique ID (UID) that is associated or correlated with a patient ID for the patient. The UID is distinct and different from the patient ID. In an embodiment, the electronic record request can include additional various patient demographic information as criteria, such as, but not limited to, a patient first, middle, and/or last name, date of birth, social security number, sex, address, telephone number, insurance information, primary care physician information, universal identifier, accession number, medical record number, and the like.

Furthermore, the electronic record request can include procedure identifiers as criteria, such as, but not limited to, CPT codes, LOINC codes, SNOMED codes, CPOE codes, procedure names, procedure codes, insurance billing codes, procedure name acronyms, procedure description, modality types, body parts, organs, limbs and/or appendages related to the procedure, and the like.

The requesting medical provider 100 can also specify a date range or number of years back for the prior medical records, such as one year, two years, five years, etc. from the current date.

The requesting medical provider can also specify the patient's appointment date, as well as an exam priority code as criteria, such as routine, STAT or RUSH, in the electronic record request.

In step 206, the electronic record request is transmitted to the external medical provider 102. In an embodiment, the electronic record request is transmitted via, for example, an electronic facsimile (e-fax), traditional fax which can then be digitized upon receipt by the external medical provider, secure electronic mail (e-mail), messaging via the HL7 format, direct message via a Health Information Service Provider (HISP), and the like. In a preferred embodiment, the electronic record request is in the form of an e-fax.

In an embodiment, the local server 114 automatically, at a predetermined time, submits an electronic record request to all or selected medical providers which are communicatively coupled to the medical network 110. The predetermined time can be configured by the requesting medical provider 100, and can be, for example, one month, two weeks, one week, etc. prior to the patient's appointment date.

For example, the requesting medical provider 100 can specify a time offset for issuing an electronic record request. In an embodiment, the time offset can be a number of calendar or business days prior to a patient's appointment date. In another embodiment, the time offset can be a delay which delays the query date based on a current date. The time offset is used by the local server 114 to determine when to issue the electronic record request.

For example, if the appointment date is 10 calendar days from the current date, and the time offset is 3 calendar days, then the local server 114 does not take any action until 7 calendar days have passed (i.e., 10 calendar days−3 calendar days=7 calendar days).

If, however, the appointment date is 10 calendar days from the current date, and the time offset is 15 calendar days, then the local server 114 proceeds with the transmitting an electronic record request, as the query date criteria has been met (i.e., 10 calendar days−15 calendar days=−5 calendar days).

In an embodiment, the local server 114 can utilize the appointment time (versus the appointment date), and compare it to a time offset based on a current time.

In an embodiment, the time offset can be in the form of calendar days, business days, a 24-hour time format, a 12-hour time format, and the like. In addition, the time offset calculation can compensate for existing holidays, scheduled closures, weekends, or known vacation of off-days for a referred physician.

In another embodiment, the time offset can be a delay. In this embodiment, the local server 114 calculates a query day based on the current date and the time offset delay (i.e., current date+time offset=query date). For example, if the time offset is “2 weeks”, then the local server 114 is configured to issue a prior medical record query two weeks after the patient appointment is generated. In another example, if the time offset delay is 3 calendar days, then the local server 114 does not take any action until 3 calendar days have passed from the current date. In another example, if the patient appointment is rescheduled, then the local server 114 calculates the query day based on the current date, the time offset delay, and the rescheduled patient appointment date.

In step 208, the external medical provider 102 receives the electronic record request, and can view the electronic record request on the portal. In an embodiment, the external medical provider 102 can view multiple electronic record requests in the portal, and can filter, query, and sort the electronic record requests by various criteria. For example, the electronic record requests can be filtered by priority code, appointment date, patient name, the requesting medical provider, and the like.

For a selected electronic record request, the system can display matching patient records, and allows the external medical provider 102 to select all or specific patient records to transmit to the requesting medical provider 100, in response to the electronic record request.

In step 210, the selected prior medical records are transmitted from the external medical provider 102 to the requesting medical provider 100. In an embodiment, the selected prior medical records are transmitted via the encrypted, secure peer-to-peer communication channels 124. In another embodiment, the selected prior medical records are transmitted via the medical network 104. In yet another embodiment, the selected prior medical records are sent via direct messaging using the HL7 or HISP format directly via the portal, or sent via e-fax using a printer driver.

In step 212, the requesting medical provider 100 receives the prior medical records, and in step 214, the prior medical records are associated with the UID of the electronic record request by the local server 114. Thus, using the prior association of the UID and the patient ID (assigned by the requesting medical provider 100), the patient identification number of the prior medical record(s) is/are updated to match the patient ID.

In step 216, the reconciled prior medical records can be held for manual review by the requesting medical provider 100, or alternatively, the reconciled prior medical records are automatically ingested and made available to the PACS 106, or any other information system, database, archive, or platform which may or may not have image viewing functionality, for review by the requesting medical provider 100. The routing of reconciled prior medical records can be determined by incoming data rules which are configured by the requesting medical provider 100 via the portal.

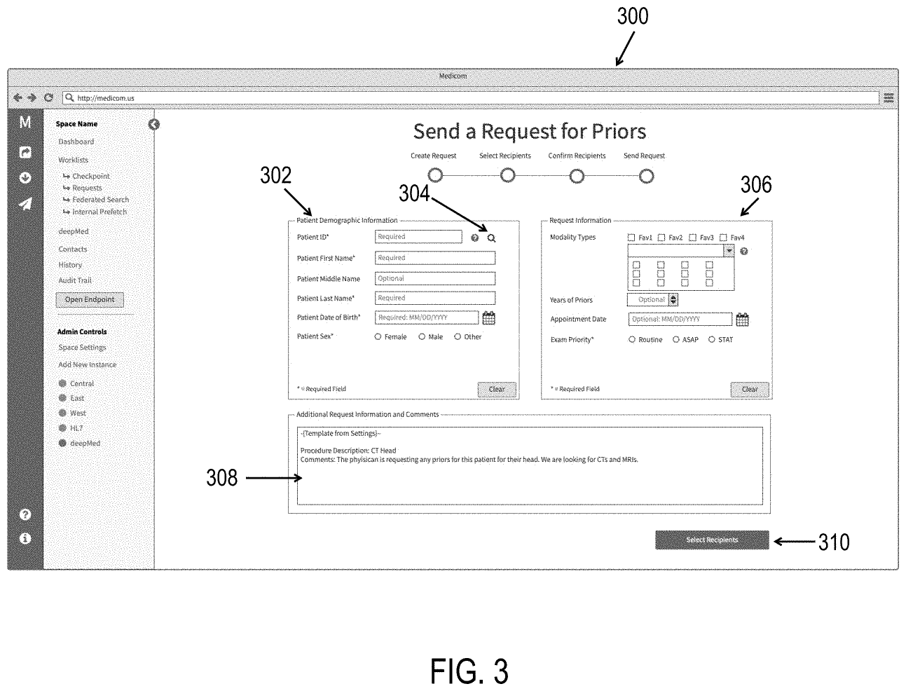

FIG. 3 is an exemplary interface 300 for creating an electronic record request, according to an embodiment of the invention. The interface 300 allows for patient demographic information 302 to be entered by the requesting medical provider 100. For examiner, the patient demographic information 300 can include fields for a patient's first, middle, and/or last name, date of birth, social security number, sex, address, telephone number, insurance information, primary care physician information, universal identifier, accession number, medical record number, and the like.

The various fields in patient demographic information 302 can automatically be populated based on the patient ID. Upon entering the patient ID and selecting the search icon 304, the local server 114 can retrieve the patient demographic information using a patient-level demographic query against a DICOM archive, patient record archive, a medical records database, and the like associated with the requesting medical provider 100. The DICOM archive can be located or communicatively coupled to the PACS 106 and/or database 108.

In an embodiment, if the patient-level demographic query does not yield any results, then the requesting medical provider 100 is alerted that the patient ID may be invalid or erroneous. For example, the patient ID field can be highlighted in red, or a pop-up message may be displayed on the interface 300.

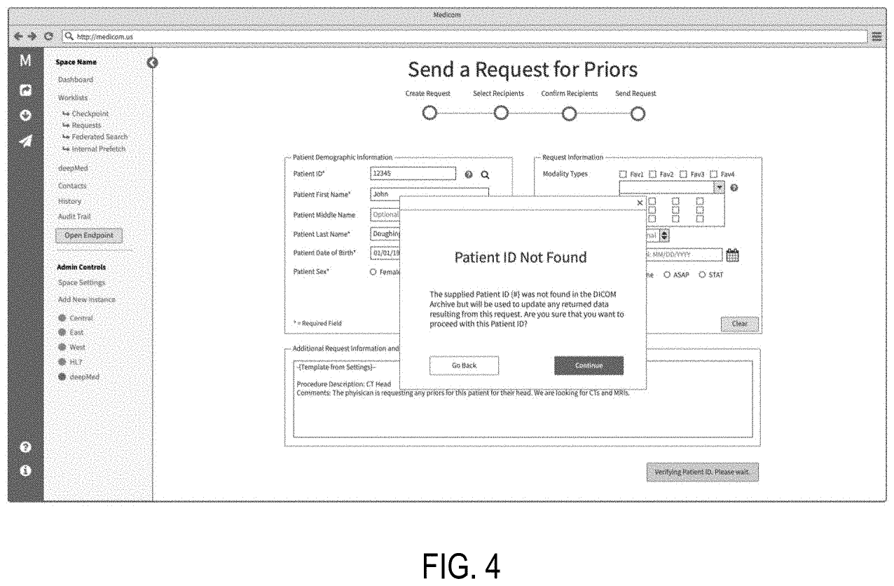

In another embodiment, the requesting medical provider 100 can elect to proceed with an invalid or erroneous patient ID, and may be required to consent to a warning before doing so, as shown in FIG. 4.

In an alternative embodiment, the patient demographic information 302 can be entered manually by the requesting medical provider 100.

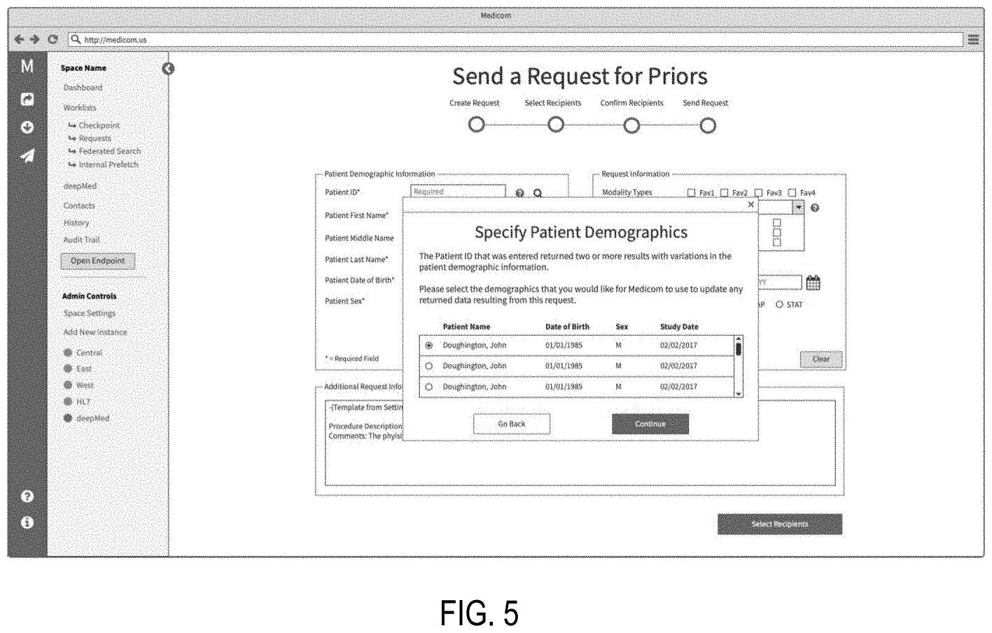

In the event that a query using the patient ID yields multiple results with differing demographic information, the interface 300 can display the multiple patient records and prompt the requesting medical provider 100 to select the patient record which they would like to utilize for the electronic record request, as shown in FIG. 5.

The interface 300 also allows for record request information 306 to entered by the requesting medical provider 100. In an embodiment, the record request information 306 can include fields such as the modality type, years of prior medical records, an appointment date, and an exam priority code. The record request information 306 allows the requesting medical provider 100 to request only prior medical records which may be relevant to the patient's appointment. For example, if the patient appointment is for a “MRI Abdomen w/o Contrast”, the record request information 306 can specify, for example, “MRI” and “Abdomen” as criteria. Thus, any prior medical records stored with an external medical provider 102 that is related to other modalities or body parts (i.e., an x-ray of a hand), would be deemed not relevant to the patient appointment, and such prior medical records would not be retrieved, or displayed for selection in order to fulfil the electronic record request.

In an embodiment, the record request information 306 can include procedure identifiers, such as, but not limited to, CPT codes, LOINC codes, SNOMED codes, CPOE codes, procedure names, procedure codes, insurance billing codes, procedure name acronyms, procedure description, modality types, body parts, organs, limbs and/or appendages related to the procedure, and the like.

In this embodiment, procedure identifiers can be translated to specific modality types by the local server 114. For example, a CPT code of “74181” is for a “MRI Abdomen w/o Contrast”, while a CPT code of “75572” is for a “CT Heart w Contrast”.

In an embodiment, patient consent is required before an external medical provider 102 can fulfill a request for prior medical records. The patient consent can be completed by the patient prior to the request, and transmitted to the external medical provider 102 along with the record request information 306. In another embodiment, the patient consent form can be automatically generated by the local server 114 upon a determination by the requesting medical provider 100 that prior external medical records may exist. The patient consent form can be transmitted to the patient for a physical or digital signature, and can be electronically transmitted back to the requesting medical provider 100 where the completed patient consent is then stored. In the event the patient consent form is completed via physical copy, the physical copy can be digitized by the requesting medical provider 100.

In another embodiment, the external medical provider 102, upon receiving a request for prior medical records, can generate a patient consent form, and transmit it to the patient for signature.

The interface 300 also allows the requesting medical provider 100 to enter additional request information 308, which can include comments, notes, procedure descriptions, and the like. For example, the local server 114 can analyze keywords and phrases from the additional request information 308 field to determine body parts, organs, limbs and/or appendages which may be relevant to the patient appointment. For example, if the additional request information 308 includes text such as “left arm”, then the phrase “left arm” can be included as criteria in the prior medical record query. In this example, any prior medical records related to a “right arm” would not be retrieved.

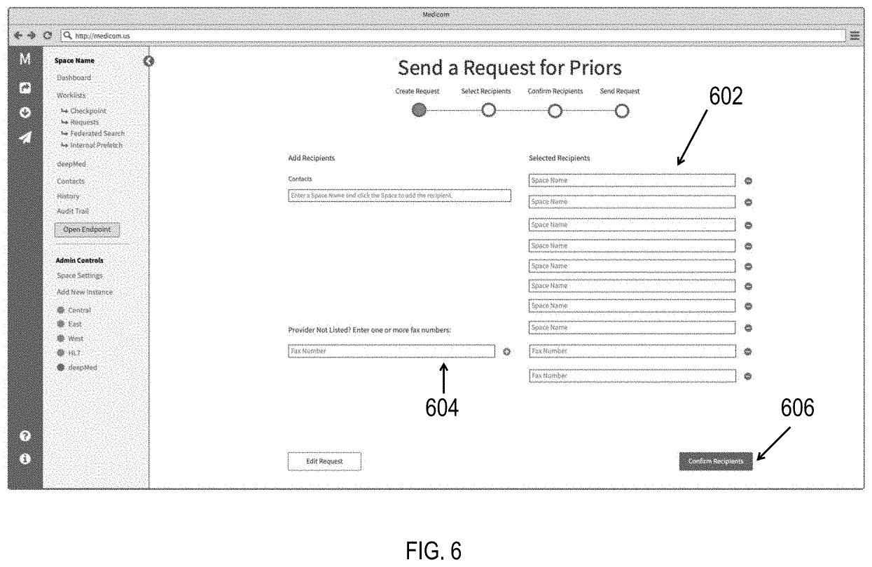

Once the patient demographic information 302 and request information 306, and optionally the additional request information 308, has been entered by the requesting medical provider 100, the “Select Recipients” button 310 is selected and the requesting medical provider 100 is shown interface 600 depicted in FIG. 6.

FIG. 6 is an exemplary interface 600 for selecting recipients for an electronic record request, according to an embodiment of the invention. The interface 600 allows the requesting medical provider 100 select external medical providers 102 which are registered with the medical network 104 by entering a provider name in the text boxes 602. As the name is typed into a text box 602, a scrollable dropdown list can appear (not shown) that is auto-populated with matching external medical provider names. If a provider name does not match any external medical providers 102 registered with the network 104, then the requesting medical provider 100 can enter a fax number into text box 604 in order to transmit a PDF version of the electronic record request via fax.

In an embodiment, the database 112 can maintain and store a list of providers which are registered, authenticated, and/or associated with the medical network 104 and/or with the medical network operator 105.

In an embodiment, external medical providers 102 can restrict access, such that they are not capable of receiving electronic record requests, or they can choose to not receive electronic record requests for specific patients, facilities, physicians, and the like. The database 112 can further include a list of permissions and privileges which have been configured by each provider.

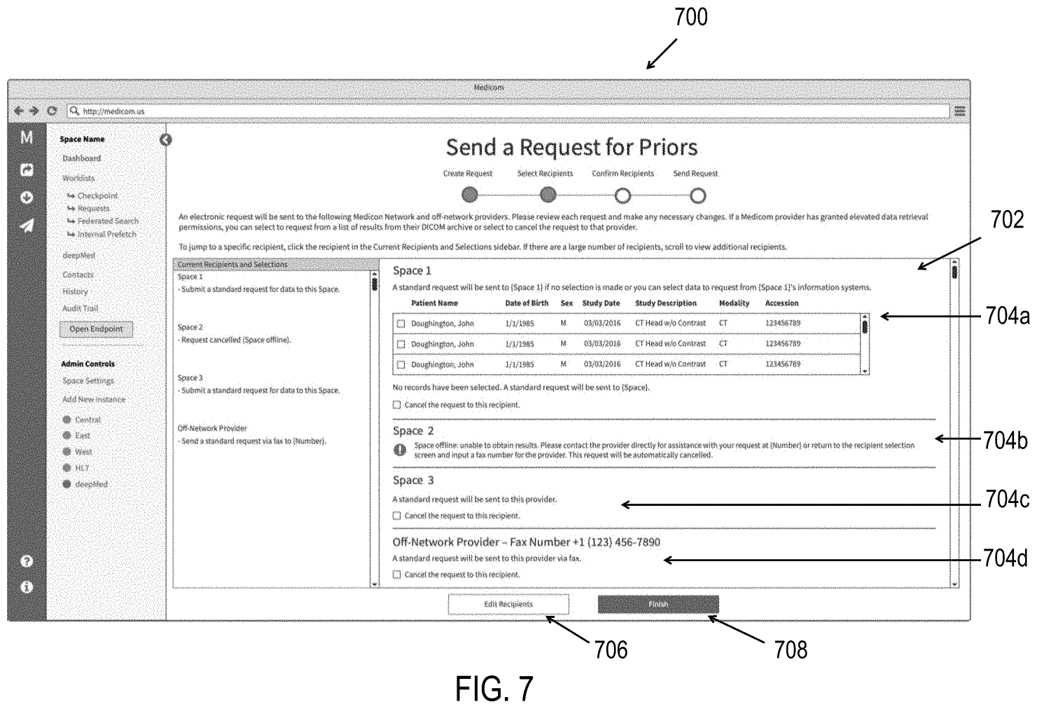

Once the requesting medical provider 100 has completed entering the recipients for the electronic record request, the requesting medical provider 100 is shown interface 700 depicted in FIG. 7.

FIG. 7 is an exemplary interface 700 for confirming an electronic record request, according to an embodiment of the invention. The interface includes a scrollable list 702 of recipients 704a-n for the electronic record request that the requesting medical provider 100 entered on interface 600 shown in FIG. 6. An electronic record request for a particular recipient 704 can be cancelled, and furthermore, the requesting medical provider 100 can select the “Edit Recipients” button 706, and is taken to the interface 600 to re-select the recipients.

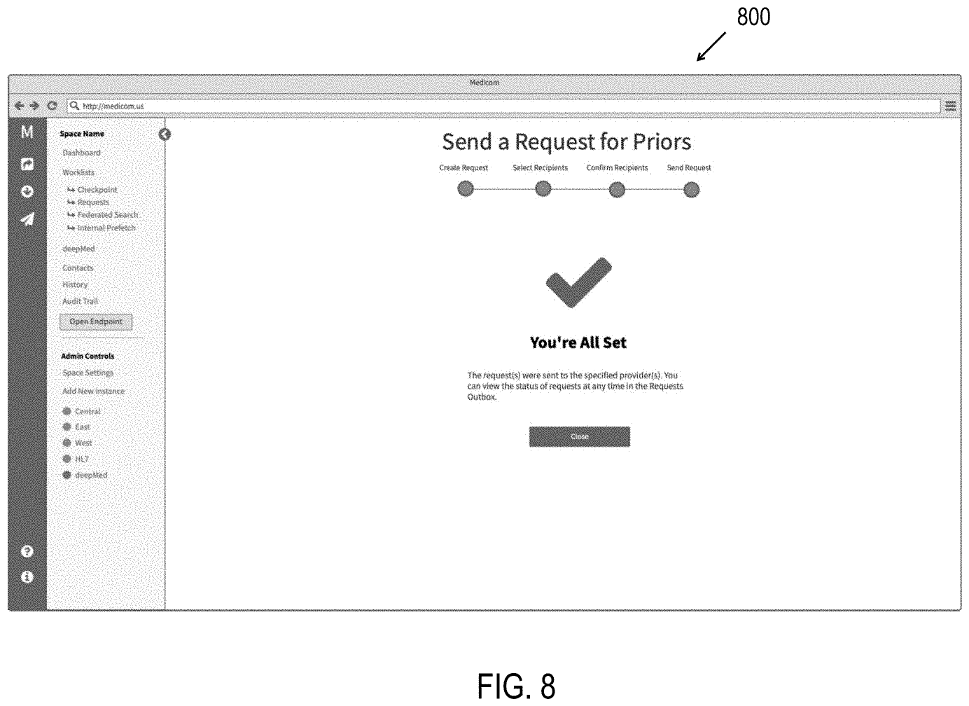

Once the requesting medical provider 100 has confirmed the recipients for the electronic medical request, the “Finish” button 708 is selected and the requesting medical provider 100 is shown the interface 800 depicted in FIG. 8.

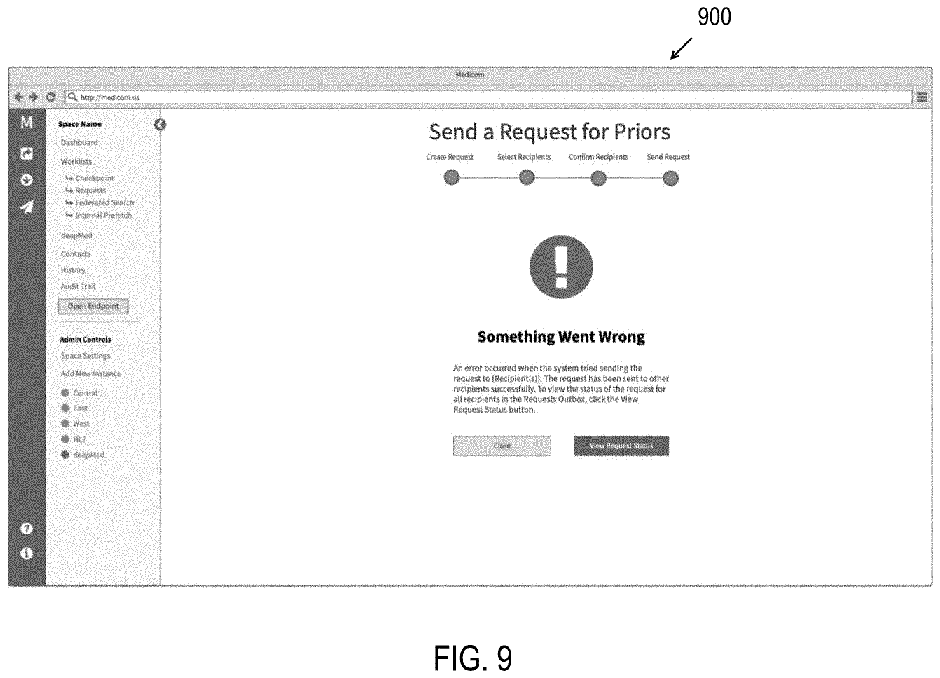

FIG. 8 is an exemplary interface 800 for displaying a success status of an electronic record request, according to an embodiment of the invention. If the electronic record request was successfully transmitted to all of the selected recipients, then the interface 800 is displayed. If, however, the electronic record request was not successfully transmitted to some or all of the selected recipients, then the interface 900 depicted in FIG. 9 is shown, indicating recipients for which there was a transmission error.

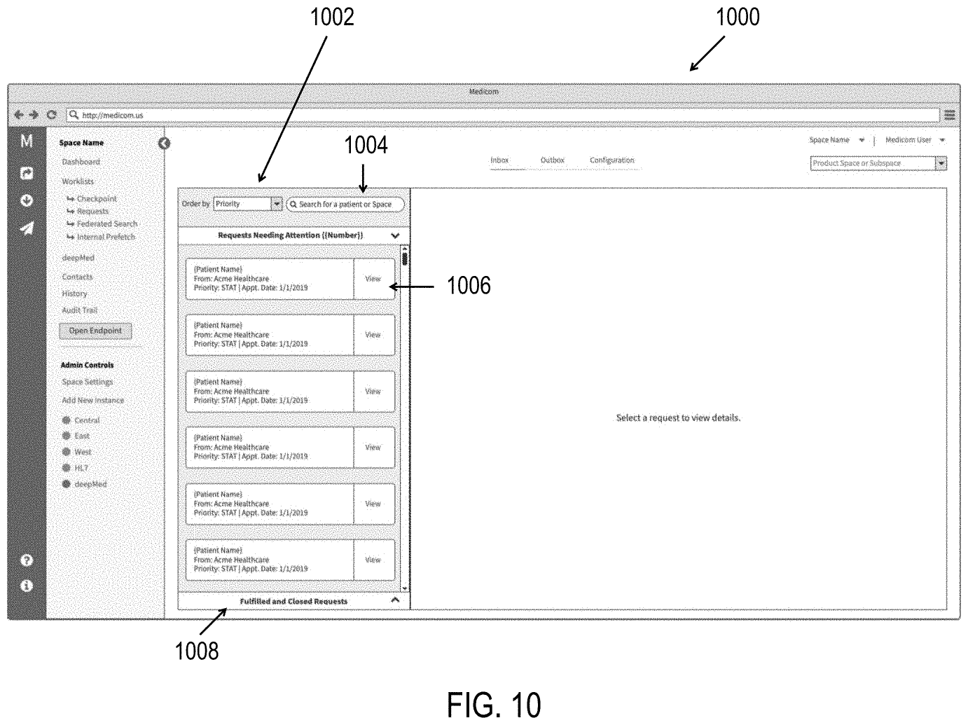

FIG. 10 is an exemplary interface 1000 displaying an inbox for received electronic record requests, according to an embodiment of the invention. The interface 1000 is used by a medical provider which received an electronic medical request. The term “external medical provider” as used herein refers to a medical provider which receives electronic medical requests. It is noted that an external medical provider can also be a requesting medical provider, and vice versa.

The interface 1000 displays a list 1002 of electronic record requests which require attention. The list 1002 can be filtered by various criteria, including, but not limited to, priority code, appointment date, electronic record request receipt date, physician, facility name, patient name, requesting medical provider, and the like.

In addition, the external medical provider 102 can enter text in text box 1004 to query the list 1002 based on keywords, names, identification numbers, codes, etc. The list 1002 can automatically update based on the text entered, or being entered as the user is typing, in the text box 1004.

The external medical provider 102 can select a particular electronic record request by selecting the “View” button 1006 which then displays additional details regarding the selected request. The external medical provider 102 can also select the “Fulfilled and Closed Requests” tab 1008 to display a list of electronic record requests which have been viewed, fulfilled, and closed.

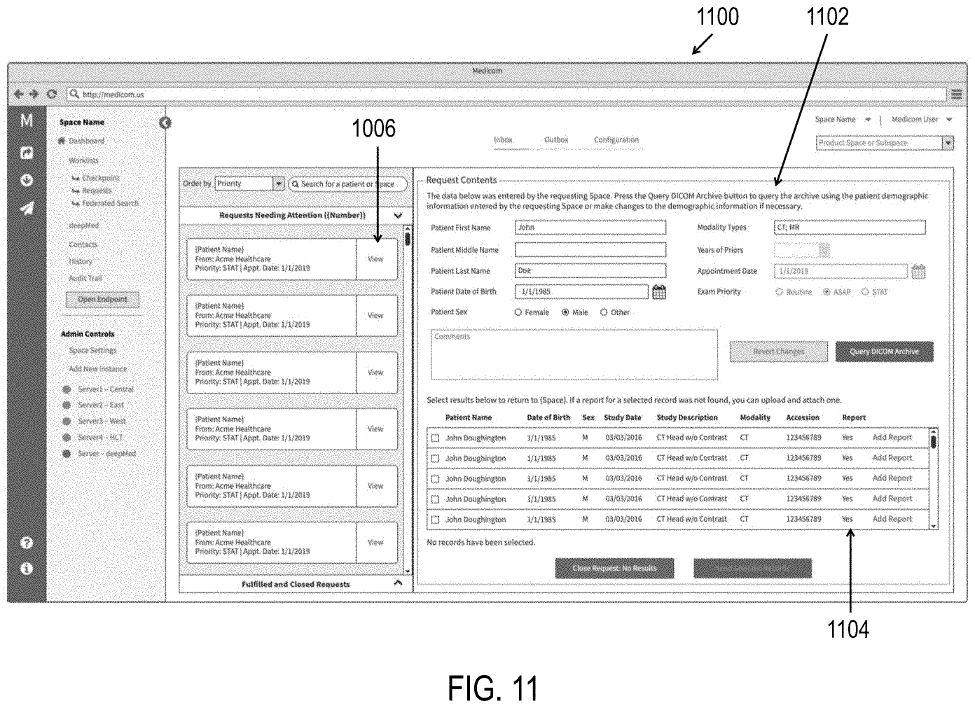

FIG. 11 is an exemplary interface 1100 displaying a selected electronic medical request 1102 according to an embodiment of the invention. Upon selecting the “View” button 1006 for a particular electronic record request, the electronic record request details 1102 are shown. The local server 116 automatically queries the database 110 of the external medical provider 102 and displays prior medical records which match the electronic record request in list 1104. In an embodiment, the list 1104 is scrollable and allows the external medical provider to manually select specific prior medical records, or all of the prior medical records, to return to the requesting medical provider 100.

In an embodiment, the local server 116 utilizes a C-FIND request which conforms to the DICOM standard in order to populate list 1104 with the matching prior medical records. The C-FIND service is an operation by which relevant patient information and medical records can be queried across disparate databases.

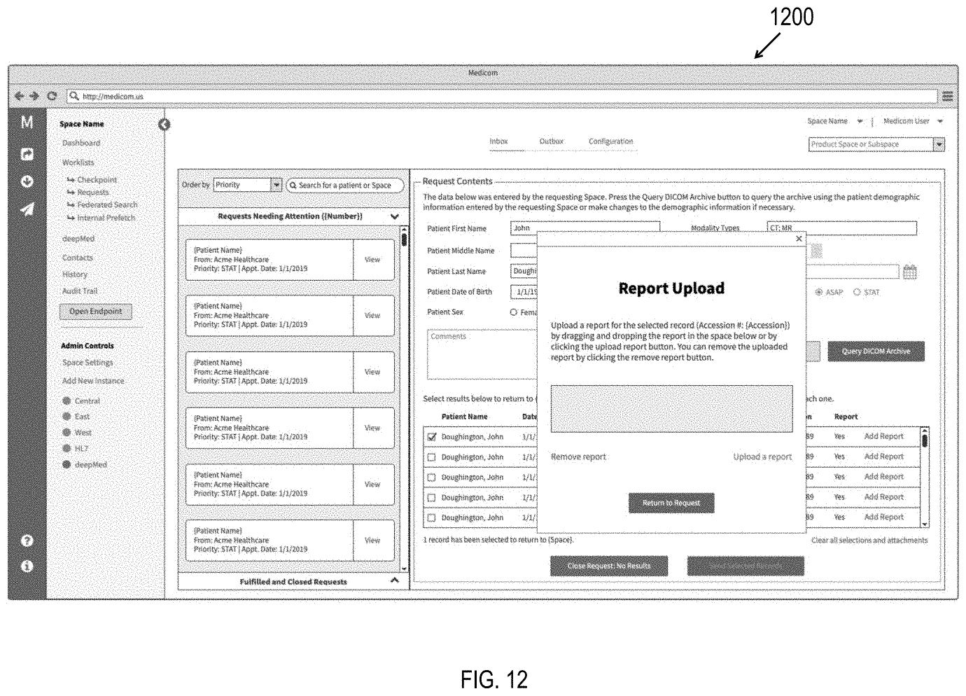

FIG. 12 is an exemplary interface 1200 for uploading files to fulfill an electronic medical request, according to an embodiment of the invention. For a selected prior medical record, the external medical provider 102 can upload various files, such as reports, notes, static images, and the like. The files can be in the form of a PDF document, as well any type of encapsulated document, a Microsoft® Word document, an Open document, an Apple® Mac page, a rich text document, a StarOffice® document, a text document, a Corel® WordPerfect document, a Google® document, an Excel file, a compressed file, an image file, a multimedia file, and the like.

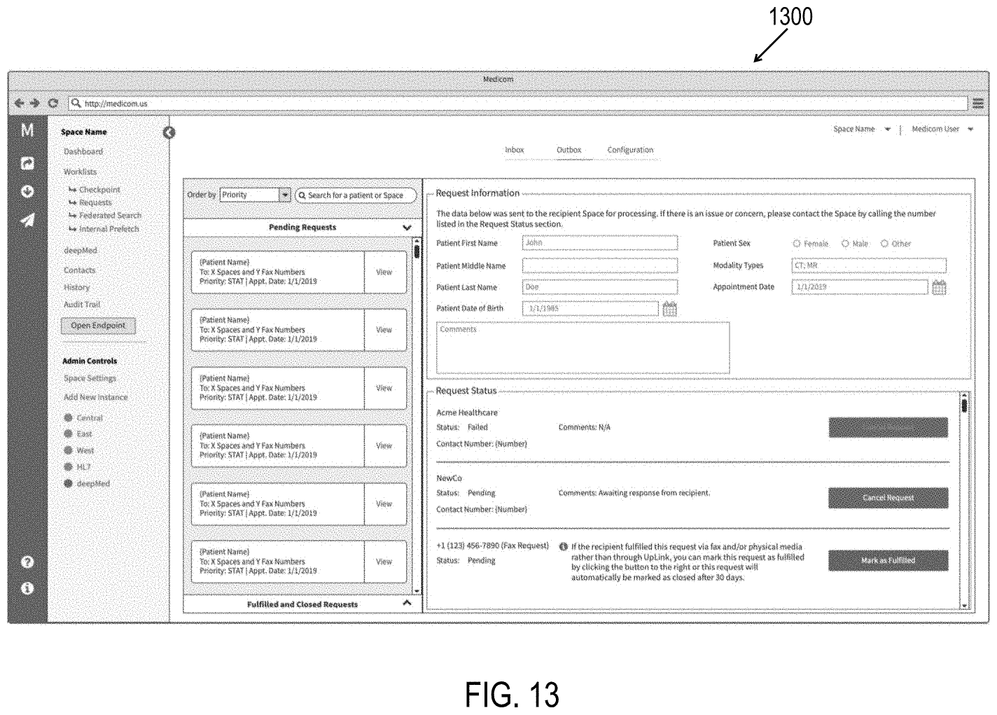

FIG. 13 is an exemplary interface 1300 displaying an outbox for fulfilled electronic record requests, according to an embodiment of the invention. For a selected electronic record request, the external medical provider 102 can cancel a particular request. Furthermore, the external medical provider 102 can mark an electronic record request as fulfilled once it has been processed and all prior medical records matching the electronic record request have been sent to the requesting medical provider 100. Such functionality exists as it allows external medical providers 102 to mark electronic record requests as fulfilled, in the event that the external medical providers provide the prior medical reports using physical media or fax to the requesting medical provider 100.

In this embodiment, if the external medical provider 100 does not manually mark an electronic record request as fulfilled after sending physical media or a fax, the electronic record request will remain in the list 1002 for thirty (30) days before being moved to a “closed: 30 day fax request timeout” status.

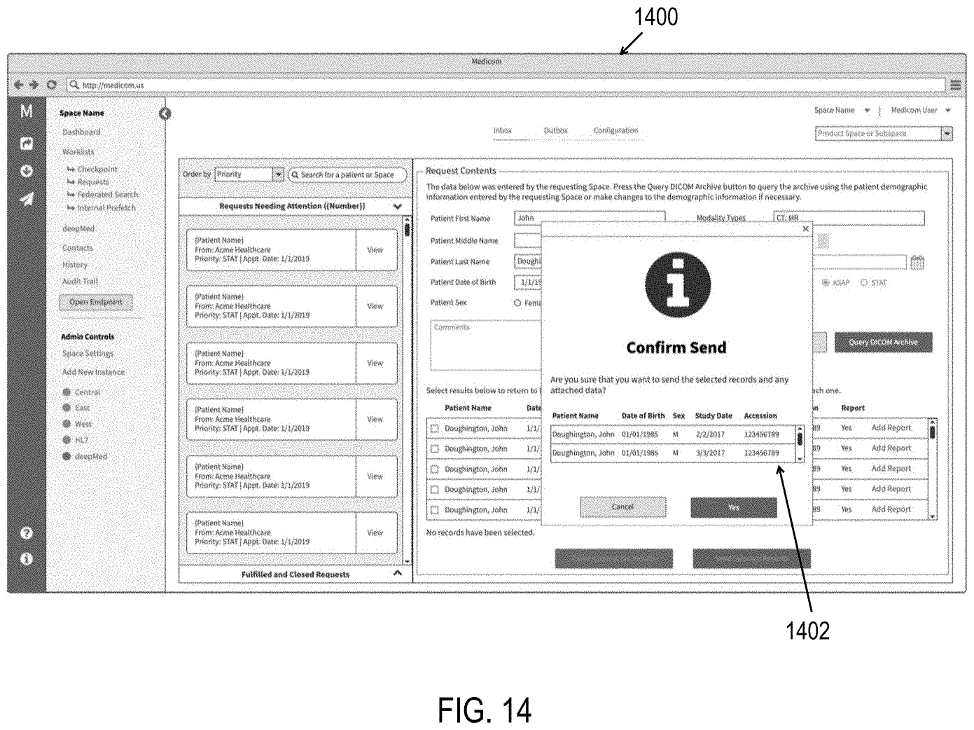

FIG. 14 is an exemplary interface 1400 to confirm a transmission of selected prior medical records 1402 to a requesting medical provider, according to an embodiment of the invention. After confirming that the selected prior medical records 1402 are to be transmitted, the selected prior medical records are sent to the requesting medical provider 100. In an embodiment, the local server 116 initiates a secure peer-to-peer communication channel 124 directly between the external medical provider 102 and the local server 114 coupled to the requesting medical provider 100. The peer-to-peer communication channel 124 is not coupled to the server 122 or to the medical network 104, or to any third-party, external, internal servers, computing devices, machines, cloud networks, and the like. The peer-to-peer communication channel 124 allows for the external medical record to be securely transmitted directly from the external medical provider 102 to the requesting medical provider 100 without any intermediary parties or systems coming into contact with the external medical record while in transit via the peer-to-peer communication channel. The external medical record is not downloaded to the server 106, or to any third-party server, for subsequent retransmission to the requesting medical provider 100.

In an embodiment, the medical network server 122, or local server 116, can encrypt the prior medical record prior to transmission by utilizing cryptographic protocols, hashing algorithms, key-based encryption, obfuscation, and the like. In this embodiment, the requesting medical provider 100 can be provided with a private key to decrypt the encrypted external medical record, where the private key can be transmitted along with the external medical record, or alternatively, can be communicated in a separate transmission via the peer-to-peer communication channel.

In an embodiment, the medical network 104, database 112, and/or medical network server 122 can store an audit trail related to the electronic record request. The audit trail can include various information, such as details related to the requesting medical provider 100 and the external medical provider 102, as well as the electronic record request date, requested patient information, technical information related to the external medical provider equipment, transmission information related to the peer-to-peer communication channel (i.e., transmission time, speed, latency, and the like), storage location information at the requesting medical provider 100, contact information of the external medical provider 102, and the like.

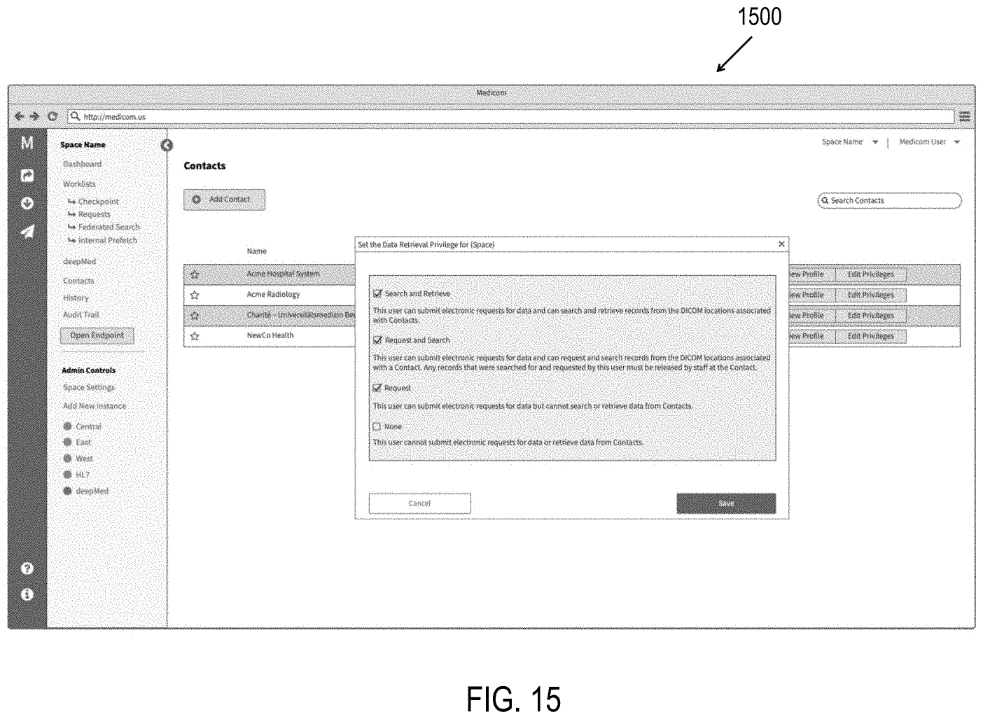

FIG. 15 is an exemplary interface 1500 for configuring permissions and privileges by the medical providers 100, 102, according to an embodiment of the invention. The medical providers 100, 102 can set various privileges and permissions for other medical providers. For example, a medical provider 100 can modify the privileges for certain medical providers 102a-n, and can selectively restrict or allow electronic record request functionality for each of the other medical providers 102a-n.

In an embodiment, the medical provider 100, 102 can assign permissions for specific users located within their organization or facility. For example, medical provider 100 may have query and retrieve permission from medical provider 102a. However, medical provider 100 may internally restrict permissions for a particular staff member to requesting medical records only. Thus, a user is always bound by the most restrictive permissions applied to their organization.

In an embodiment, there are multiple levels of permissions, listed from the most permissive to the most restrictive:

Query and Retrieve/Search and Retrieve: Allows authorized users that are members of an external provider to execute queries against a remote DICOM archive and retrieve the data. This is no restriction from the medical network operator 105 and/or local servers 114, 116 on patients or data that can be queried.

Request and Search: Allows users that are members of an authorized medical provider to execute queries against a remote DICOM archive and request resulting records following the completion of an electronic request form. A query can only be executed if the medical network operator 105 and/or local servers 114, 116 has verified that the patient data being queried exists in the requesting medical provider's information systems.

Request: Allows users to submit electronic record requests to external medical providers. Users do not need to be members of an authorized medical provider to issue requests; and in an embodiment, this permission is granted to all medical providers 100, 102 which are registered with the medical network 104.

No Request or Query: Prevents users at external medical providers from being able to request, search, or retrieve. This permission can be applied to all external medical providers who are not authorized.

The permission levels described above are not all inclusive, and those of ordinary skill in the art will appreciate that various combinations, forms, and levels of permissions can be utilized by the present invention.

While the principles of the disclosure have been illustrated in relation to the exemplary embodiments shown herein, the principles of the disclosure are not limited thereto and include any modification, variation, or permutation thereof.

What is claimed is:

1. A method for retrieving a medical record from a remote database, comprising:
scheduling a patient for an appointment by a first medical provider;
determining, by the first medical provider, if the patient has a prior medical record from a second medical provider;
transmitting, by the first medical provider, an electronic record request to the second medical provider via a data transfer network, wherein the electronic record request includes a request for the prior medical record;
transmitting, by the second medical provider, the prior medical record to the first medical provider;
receiving, by the first medical provider, the prior medical record; and
updating, by the first medical provider, a prior patient identification number associated with the prior medical record.

2. The method of claim 1, wherein the electronic record request includes a unique identification number that is associated with a patient identification number.

3. The method of claim 2, wherein the prior patient identification number is updated to match the patient identification number.

4. The method of claim 1, wherein the second medical provider transmits the prior medical record to the first medical provider via a secure peer-to-peer communication channel.

5. The method of claim 1, wherein the electronic record request includes patient demographic information and record request information.

6. The method of claim 5, wherein the record request information includes at least one criteria selected from a group consisting of a modality type, a procedure identifier, years of prior medical records requested, an appointment date for the patient, and an exam priority code.

7. The method of claim 1, wherein the first medical provider transmits the electronic record request to the second medical provider via electronic fax.

8. A system for retrieving prior medical records for a patient from a remote database, comprising:
a server communicatively coupled to both a first medical provider and a second medical provider;
a first local server coupled to the first medical provider, wherein the first local server is an instance of the server;
a second local server coupled to the second medical provider, wherein the second local server is an instant of the server;
a first medical records database coupled to the first local server;
a second medical records database coupled to the second local server; and
a portal capable of being executed on the first local server and the second local server,
wherein the first local server is capable of automatically retrieving patient demographic information from the first medical records database based on a patient identification number, and
wherein the first local server generates an electronic record request using at least one of the patient demographic information and record request information, and
wherein the first local server transmits the electronic record request to the second local server via the server.

9. The system of claim 8, wherein the first local server is capable of associating a unique identification number with the patient identification number, wherein the unique identification number is different than the patient identification number.

10. The system of claim 8, wherein the record request information includes at least one selected from a group consisting of a modality type, a procedure identifier, years of prior medical records requested, an appointment date for the patient, and an exam priority code.

11. The system of claim 8, wherein the second local server receives the electronic record request and is capable of querying the second medical records database based on at least one of the patient demographic information and the record request information to identify matching prior medical records.

12. The system of claim 11, wherein the second local server transmits the prior matching medical records to the first local server.

13. The system of claim 12, wherein the second local server transmits the prior matching medical records to the first local server via a secure peer-to-peer communication channel.

14. The system of claim 8, wherein the first local server transmits the electronic record request to the second local server via electronic fax via the server.

15. A system for retrieving a prior medical record for a patient from a remote database, comprising:
a patient scheduling system communicatively coupled to a first medical provider, wherein the patient scheduling system is capable of generating an appointment date;
a server communicatively coupled to both the first medical provider and a second medical provider;
a first local server coupled to the first medical provider, wherein the first local server is an instance of the server;
a second local server coupled to the second medical provider, wherein the second local server is an instant of the server;
a first medical records database coupled to the first local server;
a second medical records database coupled to the second local server; and
a portal capable of being executing on the first local server and the second local server,
wherein the first local server is capable of automatically retrieving patient demographic information from the first medical records database based on a patient identification number, and
wherein the first local server generates an electronic record request using the patient demographic information, record request information, the appointment date, and a unique identification number, and
wherein the first local server transmits the electronic record request to the second local server via the server.

16. The system of claim 15, wherein the first local server is capable of associating the unique identification with the patient identification number, wherein the unique identification is different than the patient identification number.

17. The system of claim 15, wherein the record request information includes at least one criteria selected from a group consisting of a modality type, a procedure identifier, years of prior medical records requested, an appointment date for the patient, and an exam priority code.

18. The system of claim 15, wherein the prior medical record includes at least one of a healthcare data, patient data, an electronic medical record, a diagnostic imaging study, a diagnostic image, a diagnostic imaging report, fitness and activity data, a patient chart, a medical opinion, a medical history, an immunization history, a surgical history, clinical information, a growth and development history, a medical encounter history, and physical examination observations.

19. The system of claim 15, wherein the first local server transmits the electronic record request to the second local server via electronic fax via the server.

20. The system of claim 15, further comprising, receiving, by the first local server, the prior medical record from the second local server, and further comprising updating a prior patient identification number associated with the prior medical record with the patient identification number.
